(19) United States
(12) Patent Application Publication
(10) Pub. No.: US 2022/0331200 A1
(43) Pub. Date: Oct. 20, 2022

(54) RESPIRATORY THERAPY VEST

(71) Applicant: KONINKLIJKE PHILIPS N.V., Eindhoven (NL)

(72) Inventors: Harold Johannes Antonius Brans, Eindhoven (NL); Kiran Hamilton J. Dellimore, Eindhoven (NL); Samer Bou Jawde, Cambridge, MA (US); Marco Baragona, Eindhoven (NL)

(21) Appl. No.: 17/689,046

(22) Filed: Mar. 8, 2022

Related U.S. Application Data
(60) Provisional application No. 63/176,551, filed on Apr. 19, 2021.

(51) Int. Cl. A61H 23/04; A61M 16/00

(52) U.S. Cl. CPC A61H 23/04; A61M 16/0006; A61H 2201/165; A61H 2205/084; A61H 2201/5007

(57) ABSTRACT

Described herein are methods, devices and systems for respiratory therapy delivered by a therapy vest. A therapy vest is provided in which an independent body fitting function is provided by use of a body fit layer or compartment(s). The body fit layer or compartment(s) may be controlled independently from the therapy layer or compartment(s), such that the fit of the therapy vest is placed on more equal footing with the therapy delivering components of the therapy vest. Other examples are disclosed and claimed.

### CROSS-REFERENCE TO RELATED APPLICATIONS

This patent application claims the priority benefit under 35 U.S.C. § 119(e) of U.S. Provisional Application No. 63/176,551, filed on Apr. 19, 2021, the contents of which are herein incorporated by reference.

### FIELD OF THE INVENTION

The subject matter described herein generally relates to a therapy vest to be worn by a patient for use in connection with treatment of respiratory conditions and certain examples of systems, methods and products relate to the fit or comfort of a therapy vest.

### BACKGROUND OF THE INVENTION

Airway clearance therapy, such as high frequency chest wall oscillation (HFCWO), is an airway clearance approach that has been demonstrated to help clear the lungs of secretions in patients with different types of lung disease (e.g., cystic fibrosis (CF), bronchiectasis, and chronic obstructive pulmonary disease (COPD)). The goal of HFCWO is to loosen mucus that has pooled in the airways so that patients can clear it more easily. Therapy is usually performed once or twice daily, with a typical duration of 30 minutes.

To perform or implement airway clearance therapy, devices such as vests are used and can create an oscillating pressure on the chest. This oscillating pressure can be accomplished by using a pneumatic mechanism, such as an oscillating airflow into an inflatable compartment of the vest or via another mechanism, such as a physical mechanism or actuator that provides oscillation.

In a vest that uses an oscillating airflow into an inflatable compartment, by way of example, the vest may be connected with two tubes to an airflow generator. Personalization of a therapy session can be done by setting the air flow, oscillating frequency and time. In order to secure a decent body fit, next to inflating the vest compartment, several sizes of the vests are available (e.g., from child to adult sizes).

### SUMMARY OF THE INVENTION

Various embodiments provide methods, devices and systems for respiratory therapy delivered by a therapy vest. In an embodiment, a therapy vest is provided in which an independent body fitting function is provided by use of a body fit layer or compartment(s). The body fit layer or compartment(s) may be controlled independently from the therapy layer or compartment(s), such that the fit of the therapy vest is placed on more equal footing with the therapy delivering components of the therapy vest.

In summary, one embodiment provides a therapy vest, comprising: a vest body that includes: one or more body fit compartments; and one or more therapy compartments; wherein one or more of the one or more body fit compartments and one or more of the one or more therapy compartments are configured to be independently controllable.

Another embodiment provides a system, comprising: an airflow generator; and a therapy vest, comprising: a vest body that includes: one or more body fit compartments; and one or more therapy compartments; wherein one or more of the one or more body fit compartments and one or more of the one or more therapy compartments are configured to be independently controllable using airflow from the airflow generator.

A further embodiment provides a system, comprising: a controller configured to obtain one or more body fit parameters; and a therapy vest, comprising: a vest body that includes: one or more body fit compartments; one or more sensors; and one or more therapy compartments; wherein the one or more body fit compartments are inflated by the airflow generator based on one or more of the one or more body fit parameters and data obtained from the one or more sensors.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE DRAWINGS

FIG. 1 illustrates an example therapy system in accordance with an embodiment.

FIG. 1A illustrates an example therapy vest and airflow generator in accordance with an embodiment.

FIG. 2 illustrates example configurations of therapy vest components in accordance with an embodiment.

FIG. 3A illustrates examples of body shapes and body fit compartments in accordance with an embodiment.

FIG. 3B illustrates an example method of controlling a therapy vest in accordance with an embodiment.

FIG. 4 illustrates an example control system in accordance with an embodiment.

### DETAILED DESCRIPTION OF EMBODIMENTS

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the claims, but is merely representative of those embodiments.

Reference throughout this specification to “embodiment(s)” (or the like) means that a particular feature, structure, or characteristic described in connection with the embodiment is included in at least one embodiment. Thus, appearances of the phrases “according to embodiments” or “an embodiment” (or the like) in various places throughout this specification are not necessarily all referring to the same embodiment.

As used herein, the singular form of “a”, “an”, and “the” include plural references unless the context clearly dictates otherwise. As used herein, statements that two or more parts or components are “coupled,” “connected,” or “engaged” shall mean that the parts are joined, operate, or co-act together either directly or indirectly, i.e., through one or more intermediate parts or components, so long as a link occurs. Directional phrases used herein, such as, for example and without limitation, top, bottom, left, right, upper, lower, front, back, and derivatives thereof, relate to the orientation of the elements shown in the drawings and are not limiting upon the scope of the claimed invention unless expressly recited therein. The word “comprising” or “including” does not exclude the presence of elements or steps other than those described herein and/or listed in a claim. In a device comprised of several means, several of these means may be embodied by one and the same item of hardware. The term “about” or “approximately” as used herein includes conventional rounding of the last significant digit.

Furthermore, the described features, structures, or characteristics may be combined in any suitable manner in one or more embodiments. In the following description, numerous specific details are provided to give a thorough understanding of example embodiments. One skilled in the relevant art will recognize, however, that aspects can be practiced without one or more of the specific details, or with other methods, components, materials, etc. In other instances, well-known structures, materials, or operations are not shown or described in detail to avoid obfuscation.

HFCWO therapy is mainly performed at home. For the devices with an airflow generator, patients are limited in movement and need to stay in a fixed location due to the tubing that connects the airflow generator and the therapy vest. The use of portable devices creates more freedom for the patient, but are limited by the weight of the vest and the available power. While HFCWO vests have been used for decades, therapy-related aspects such as tailoring a vest to fit a patient comfortably have not been a common focus.

One aspect regarding HFCWO therapy is the adherence of the patient to the therapy. It has been reported that only 35% of the patients have a high adherence (≥80% of prescribed daily use). In contrast, self-reported data shows a high adherence of 65%. It has therefore been concluded that there is a large overestimation of self-reporting adherence. Further, it has been reported that persistent adherence over time is problematic, which may be due in part to social cognitive variables such as self-confidence and perceived concerns.

The low adherence to HFCWO therapy has gained interest from clinicians, but improvement is not straightforward. The standard prescription for over 20 years is 30 minutes twice a day. The history of this is based on the similarity to manual chest physiotherapy, and may not have any relevance to what patients actually need (i.e., it is not evidenced based). Adapting the therapy itself has been proposed to increase patient adherence, and although this approach might help, the discomfort of the therapy will not be reduced or directly addressed.

An embodiment addresses the problems of conventional approaches and systems, such as poor adherence to HFCWO vest therapy, by providing better body fit for the vest. An embodiment provides a personal fit of a therapy vest by separating the functions of fitting the vest to the body and performing oscillation therapy. Embodiments accommodate a large range of body shapes, body positions, genders, body mass index values (BMI, a ratio between body mass and square of body height) and body shape index values (BSI, ratio between mass and cube of body height). Various embodiments not only increase the comfort for the vest wearer but may also have an impact on the settings of the therapy, e.g., reducing the settings of parameters like pressure and time that are required may be implemented given better adherence to therapy.

The description now turns to the figures. The illustrated example embodiments will be best understood by reference to the figures. The following description is intended only by way of example and simply illustrates certain selected example embodiments representative of the invention, as claimed.

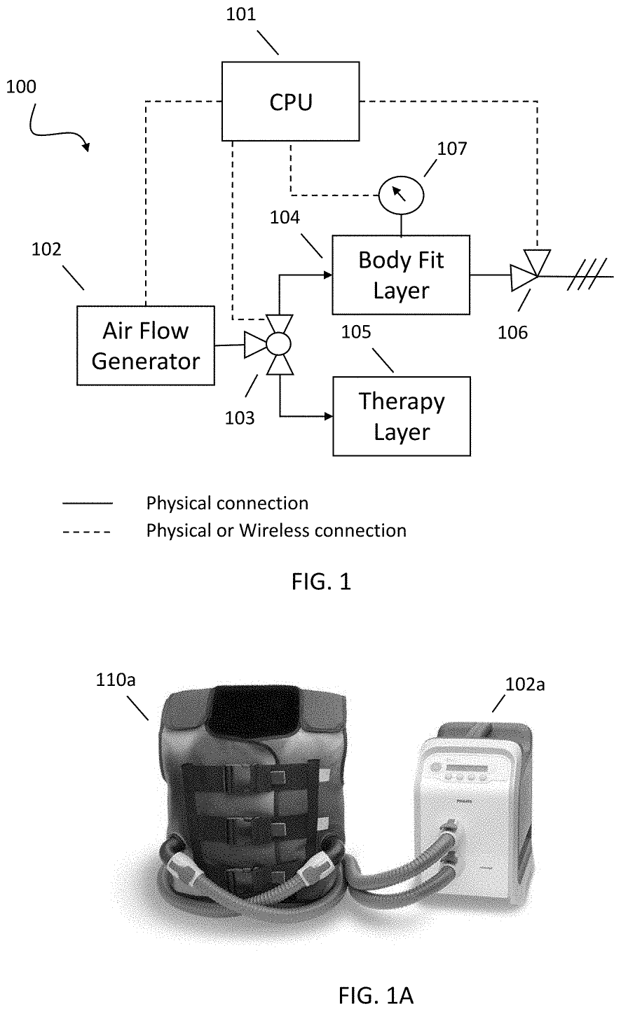

Referring to FIG. 1, an embodiment provides a system 100 that separates the functions of obtaining a personalized fit of a HFCWO vest to the patient and delivering therapy to the patient by separating one or more body fit compartments or layers 104, for example one or more inflatable layers, from one or more therapy compartments or layers 105, for example one or more oscillating layers. This permits a vest to be fitted to a patient without interfering with the delivery of therapy.

In an embodiment, body fit compartments or layers 104 and therapy compartments or layers 105 are provided in a fashion such that these compartments may be controlled independently. In an embodiment that utilizes pneumatic body fit layer(s) 104 and therapy layer(s) 105, an air flow is generated by an air flow generator 102, e.g., a pump system, for inflation of the layers 104, 105. In this configuration, each layer 104, 105 contains one or more inflatable compartments and has one or more pressure sensors 107 and one or more relief valves 106. In an embodiment, one or more selection valves 103 allow for independently controlling the inflation of an inflatable body fit layer 104 and a therapy layer 105. Regulation or adjustment of a body fit layer 104 and a therapy layer 105 is controlled by a central processing unit (CPU) 101, which may be integrated into the vest along with a power source (a battery) or provided via another device in communication with the vest or another system component.

In an embodiment, the system 100 as shown in FIG. 1 may be implemented in a vest body 110a with an airflow generator 102a, as shown in FIG. 1A. In an embodiment, the airflow generator 110a provides, e.g., via tubing, airflow to the vest body 110a which in turn includes a body fit layer or compartments and a therapy layer or compartments, e.g., as shown in FIG. 1.

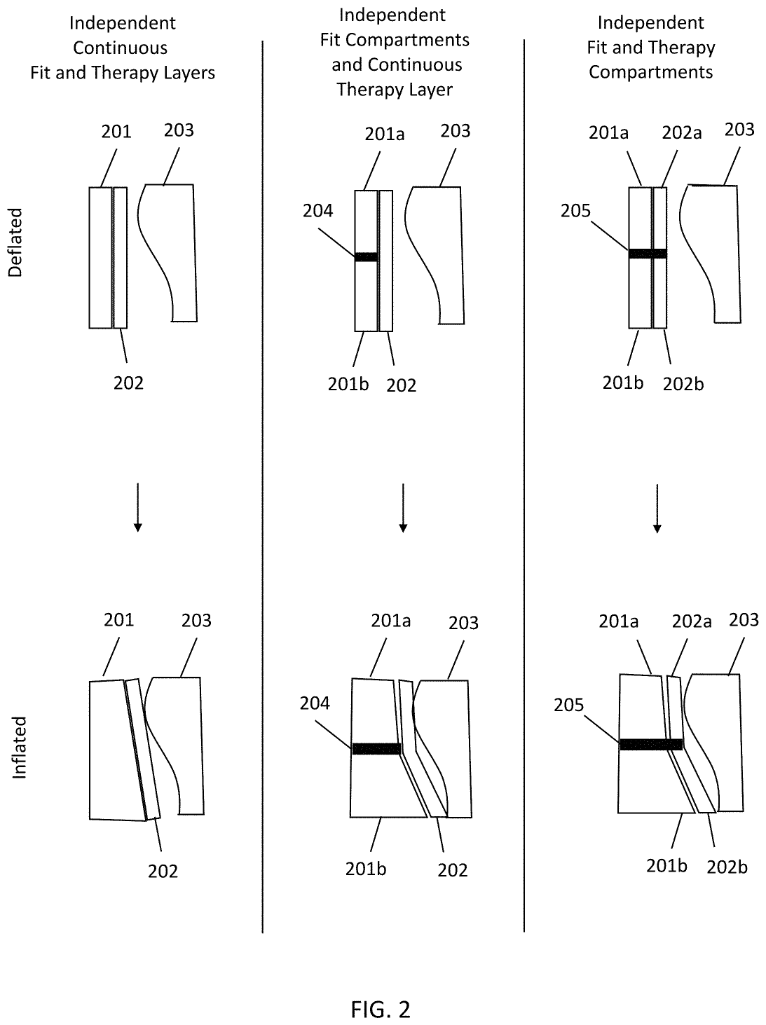

FIG. 2 illustrates an example, in cross section, of the difference in fit achievable between one or multiple compartments, both in deflated (top row of FIG. 2) and in inflated status (bottom row of FIG. 2). As illustrated in FIG. 2 (first column), using independent continuous body fit layer 201 and therapy layer 202 allows for separate, independent and more precise control of body fit with respect to a patient 203, e.g., patient's torso, particularly in a pneumatic vest. When the vest is inflated (bottom left of FIG. 2), the body fit layer 201 can be inflated with independent control with respect to the therapy layer 202, allowing the body fit layer 201 to accommodate the patient 203 more readily.

As illustrated in FIG. 2 (second column), by using separate inflatable and controllable compartments 201a, 201b in the body fit layer 201, each compartment 201a, 201b can be inflated until it conforms to a specific body area of the patient 203. Thus, when inflated (bottom center of FIG. 2), compartments 201a and 201b may be differentially inflated or fit to the patient 203. In an embodiment, a separator 204, which may include controllable components, is provided to restrict airflow between the compartments and provide a physical delineation there-between, allowing finer control of the body fit. The separator 204 may include components such as a controllable valve to selectively allow fluid communication between compartments 201a, 201b, or other components, e.g., a pressure sensor, a passive valve such as a check valve, two way valve, etc.

Additionally, as illustrated in FIG. 2 (third column), using multiple body fit compartments 201a, 201b and multiple therapy compartments 202a, 202b, the settings of corresponding compartments 201a, 201b, 202a, 202b can be coordinated or aligned, depending on the need for that specific patient 203. This permits an embodiment to more finely control body fit in the inflated condition (bottom right of FIG. 2).

In an embodiment, because separate controllable compartments or layers are supplied in the body fit layer, such as independently inflatable compartments 201a, 201b, each compartment 201a, 201b, can be controlled until it is nicely conforms to a specific body area. If also separate controllable therapy compartments are used, such as independently inflatable compartments 201a, 201b, the settings of all corresponding compartments (body fit and therapy) can be aligned. As with various other embodiments, this alignment or control may take place dynamically during therapy or may be applied at the onset of a therapy session.

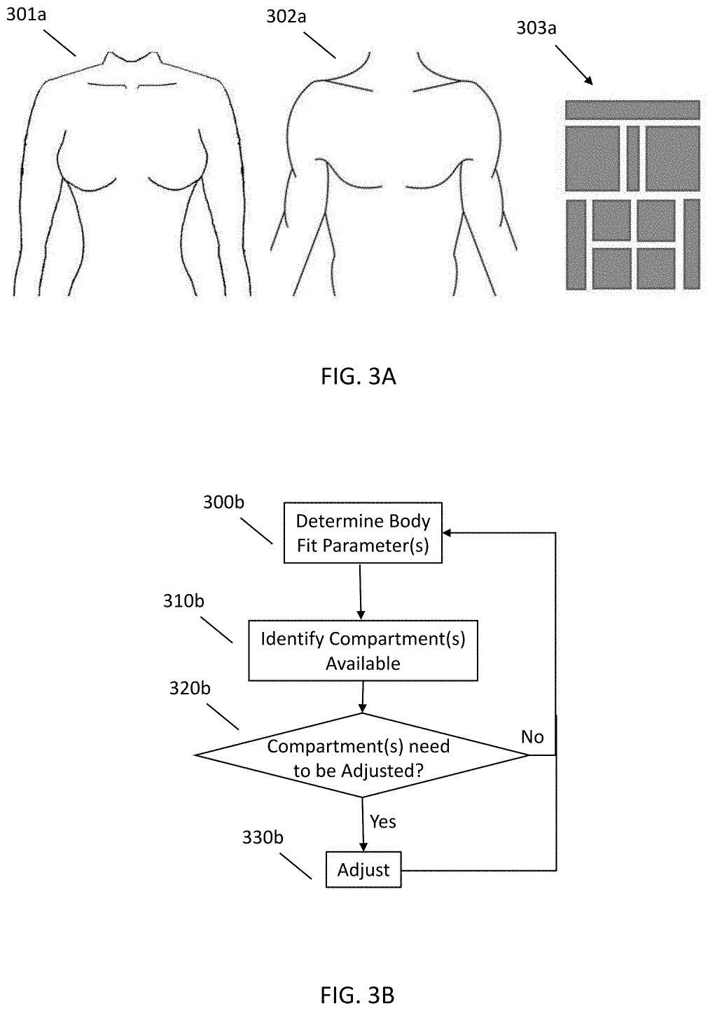

As shown in FIG. 3A, patient specific torso differences, such as shown at 301a, 302a, can be categorized and fit accordingly by using a plurality of body fit compartments, collectively indicated at 303a. This permits an embodiment to adjust or control the compartments 303a, e.g., inflating or deflating each to an individualized amount, in order for the therapy vest to have a good fit and be tailored to the need of the patient.

A schematic representation is given in FIG. 3A of one example arrangement of body fit compartments 303a. By way of non-limiting example, a certain patient 301a may have an anatomy that is associated with a predetermined or dynamically determined adjustment or control of the body fit compartments 301. Specifically, patient 301a may, for example, be most comfortable when the vest compartments mapped or located within the vest in the upper and middle chest region are inflated in an unequal manner, e.g., less in the upper chest region as compared to the middle chest region. In an embodiment, the body fit compartments 301 may be provided into areas of the vest that are mapped or located with areas that are associated with anatomical variations in patient populations associated with different fits, allowing for differential adjustment of each compartment or groups or subsets of compartments to fit the patient.

In an embodiment, body fit parameters comprise data that is used to control the layers or compartments of the vest. For example, a body fit parameter may be data derived from a categorization of different patient populations and used to allow for gross fitting adjustments to be automated initially. For example, patient specific torso differences can be categorized and related to body fit parameters used in controls, adjustments, or settings that control the body fit compartments 301a, e.g., modify their inflation.

Examples of body fit parameters that may be utilized to adjust one or more body fit compartments or a body fit layer, e.g., body fit compartments 301a, include static and dynamic body fit parameters. Examples of static body fit parameters include gender and body shape parameters, which may be obtained via a variety of mechanisms, such as obtaining numeric values (e.g., BMI or BSI, age, weigh, height, abdomen girth, shoulder girth, armpit-hip and shoulder-hip tape measurements, etc.), which generally relate to body shape. Additional or alternative sources include for example body scan data, which provides granular information about the specific patient's shape. In an embodiment, a model such as a 3D model may be formed based on data inputs, for example body measurements and/or image data, for example as described in patent application number PCT/EP2020/064044, published as WO2020234339A1, filed 20 May 2020 and having the title “Estimating a surface area and/or volume of a body or a body part of a subject,” the contents of which are incorporated by reference herein. In an embodiment, a combination of the foregoing may be used. For example, static body fit parameters may be obtained by a user inputting the values (e.g., gender indication, BMI, BSI, etc.), obtained from an external system (e.g., a body scanner that outputs a body shape categorization for the patient, such as shapes associated with a torso including but not limited to a trapezoid, triangle, inverted triangle, rectangle, oval, etc., or other external system that supplies patient data, such as a vest design tool or software program that indicates a vest size or body fit parameters based on a model, body shape, size, posture or use position, patient reported comfort, etc.), or a suitable combination of the foregoing. Static body fit parameters may be used, e.g., by CPU 101, to adjust the body fit layer or body fit compartments differently. For example, if supplied with static body fit parameters, the CPU 101 may adjust the body fit compartments, e.g., body fit compartments 303a, to accommodate the general body shape of the patient, body composition of the patient, e.g., due to tissue differences (muscle tissue, adipose tissue, etc.) associated with body shapes or compositions, and the like. For example, certain tissues have specific characteristics, such as level of compression and sensitivity, which may be used to adjust the compartments 303a.

Examples of dynamic body fit parameters include body position data. For example, an initial body fit parameter may indicate that the patient is currently reclining, whereas a subsequently obtained body fit parameter may indicate that the patient is sitting upright. During a therapy session, a patient may be lying down, sitting upright with the patient's back against a chair, reclining, etc. Further, a patient may transition through different body positions during therapy, e.g., if a patient adjusts position, changes posture, is using a mobile vest, etc. A patient could be less or more mobile due to a condition or because the type of therapy vest used, e.g., certain HFCWO devices are not mobile (e.g., are tethered to an airflow generator, as described herein).

To account for different scenarios, an embodiment is capable of taking into consideration dynamic body fit parameters such as body position, allowing for adjustment of the compartments to allow or dynamically accommodate changes to body position. It is noted here that the categorization of fit parameters as static or dynamic is for descriptive purposes only, and it is possible to consider a given body fit parameter as either static or dynamic, depending on the circumstance, e.g., a dynamic body position parameter may be considered static or relatively unchanging if the patient is always in a particular body position during therapy.

The differences in use contexts conventionally result in a poor personal fit of a standardized HFCWO vest to a patient. By defining an independently controllable body fit layer or compartments, e.g., multiple inflatable vest compartments, the body fit layer or compartments can be inflated independently of therapy layer or compartments to meet the specific needs of the patient and the context, e.g., change in position or posture.

Referring back to the example illustrated in FIG. 1, a pneumatic vest may utilize an airflow generator 102, including a pump system, in combination with valves, e.g., selection valves 103, to control or adjust the inflatable body fit layer or compartments. Controlling this inflation can be accomplished by having one or more pressure sensors, e.g., pressure sensor 107, that provide an indication of inflation level. For example, pressure sensors may be located between each body fit compartment, e.g., compartments 201a, 201b as in the example of FIG. 2, and the patient 203 or a vest layer proximate to the patient 203. One or more relief valves, e.g., relief valve 106, can be used to decrease the pressure in the body fit layer or compartments. In this way, the therapy vest can be adjusted to the body to meet the individual needs of the patient.

Input data (body fit parameters) for the setting(s) to be applied to the body fit layer or compartments can be obtained in a variety of ways. For example, body fit parameters may be obtained by using retrospective data (specific to the patient's history or otherwise, e.g., applicable to a patient population), data gathered during the initiation of therapy (e.g., a visit to a respiratory therapist), or data gather dynamically, e.g., via one or more pressure sensors or other suitable sensor(s) supplied in the vest to detect areas of proper or improper fit during therapy. It is noted that dynamic body fit parameters may be supplied via patient or therapist input, e.g., if the vest or system component is supplied with an input element to indicate areas or types of proper or improper fit, etc. As with other data, retrospective or historical data can have different origins, such as body shape analysis (e.g., via a 3-D laser body scan obtained from an external system such as a laser or other body scanner), therapy intake visit where data are collected, from historical vest settings for a given patient, patient population, patient electronic medical records, etc.

In an embodiment, a pneumatic therapy layer is provided as a separate inflatable inner layer connecting to the airflow generator, which accomplishes the oscillating function of the vest. This therapy layer, similar to the body fit layer, may be one compartment or layer or multiple compartments or components covering the torso. In a multiple compartment or component configuration, the compartments or components are separated by valves, similar to the body fit layer or compartments. Control of the oscillating function can be separated from the body fit function, e.g., by use of a selector valve. Due to its small volume compared to a current size of vest therapy layer or compartment, not much flow is needed to apply sufficient oscillation via the therapy layer.

In an embodiment, the settings, such as valve selection, pressure, etc., used to adjust or control the body fit layer or components, the therapy layer or components, or a combination thereof, may be recorded as settings. The settings from both body fit and therapy layers can be saved for subsequent reuse or application to a new, similar patient, e.g., based on body type, position of use, etc. These settings data can be used by the respiratory therapist to define in more detail future therapy scenarios for the patient. A variety of settings may be used, depending on the context. For example, in addition to body shape and position, an embodiment may provide settings suitable for different scenarios such as early morning therapy, middle of the day therapy, high or minor load therapy depending on the patient's daily condition, adjustments to therapy based on better body fit (such as lower power oscillation), etc.

Referring now to the example method illustrated in FIG. 3B, in view of the foregoing it can be appreciated that a component, e.g., CPU 101 of system 100, may be configured to adjust the body fit of a therapy vest to suit a variety of scenarios. In the example shown in FIG. 3B, a program executed by CPU 101 may implement a method including obtaining or determining body fit parameters as shown at 300b, e.g., body shape, body position, posture, etc., as sensed by pressure or other sensors, input by a therapist or patient, obtained from an external system, or the like. If more than one compartment or layer is provided, the method may include identifying available compartments or layers that can be adjusted or controlled in view of the body fit parameters, as indicated at 310b.

In view of the body fit parameters, the method may adjust the compartments of the body fit layer, e.g., via operation of a selection valve or valves that controls the inflation of the body fit compartments, controlling (passively or actively) valves that separate body fit compartments, etc., as shown at 320b. If no adjustment is presently needed (e.g., a previous or initial adjustment is still adequate), then the method may loop back to monitor body fit parameters. At 320b, a variety of mechanisms may be used to determine if one or more compartments need to be adjusted. For example, data obtained from pressure sensors may be used (e.g., compared to a threshold or acceptable range or baseline value(s), data may be obtained from a user input (such as the patient or therapist), or the like.

After one or more adjustments have been supplied, as indicated at 330b, the method may again loop back to consider the body fit parameters, e.g., in a scenario where one or more dynamic body fit parameters is taken into account to adjust the vest during the therapy. In another example, the method may end, e.g., if an initial fitting of the vest is all that is required such as in a static therapy session. As a further example, the method may determine body fit parameter(s) at 300b based on therapy effectiveness or feedback data, e.g., a patient or therapist's rating of clearance due to therapy using particular settings of body fit, which may be obtained via a mobile application or a connected computer system and used to maintain a current fit or adjust the fit of the vest.

It will be appreciated by those having ordinary skill in the art that while the above examples in part focused on a pneumatic implementation, other mechanisms and material components may be utilized to accomplish an improved body fit according to these illustrated example embodiments. For example, one or more pneumatic components, e.g., for application of therapy, may be replaced by mechanical or electroactive components. In a non-limiting example, in a mobile or semi mobile vest configuration, actuators may be used for performing oscillation or other therapy application, e.g., use of a actuator (e.g., magnetic, electrical, electroactive or piezoelectric) may take the place of a pneumatic therapy layer.

In an example embodiment, the airflow generator 102 of FIG. 1 may be omitted and inflating of the layer(s) or compartment(s) may be performed by way of a compressed gas canister (e.g., air, carbon dioxide, nitrous oxide, etc.), which may be disposable or refillable, an embedded fan integrated into the vest, a manual pump that supplies air, etc. In an embodiment, one layer may be inflated, e.g., body fit layer, whereas another layer may not be inflated, e.g., therapy layer.

The usability of a HFCWO vest may increase with an embodiment that is free from a pneumatic system component such as an airflow generator typically used to provide the therapy as the patient is no longer tethered to the airflow generator. Different user scenarios are possible, each of them suited to different type of patient needs (or conditions).

In a first example scenario, an embodiment is implemented as a non-portable system. Here, a user is tethered to an airflow generator that inflates the vest (body fit layer or compartments and therapy layer or compartments) and provides therapeutic oscillations, where both functions, body fit and oscillation, are performed by the airflow generator. For patients with low mobility (or in need of heavy therapy settings), this is configuration may be preferred.

In a second example scenario, an embodiment is implemented as a semi-portable system. This system is applicable to patients who are mobile in their home setting. In an embodiment, at the start of therapy, the patient can inflate the vest to fit appropriately, e.g., by selecting settings, manually inflating the vest, etc., and thereafter disconnect the vest from the air flow generator and start the therapy. In such a configuration, a therapy layer or compartment type that uses an actuator, e.g., electrical actuator, may be appropriate. The patient can then freely move around the home and perform other activities. In an embodiment, the use of a pneumatic or airflow based layer to deliver therapy may be facilitated by allowing for modularity in the system components. For example, a reversibly attachable airflow generator may be provided as a separate unit or module, such as in a backpack or carrying case, which may then be transported by the patient in mobile settings for attachment to the vest for therapy while making it easier to carry.

In a third example scenario, an embodiment is provided in a portable system. Here, a conventional or standard airflow generator may not be used and one or more of the body fit layers as well as therapy layer (e.g., oscillating compartments) need to be provided without the assistance of a standard air flow generator. For example, in an embodiment a battery may be supplied to power one or more components, such as a fan for inflation of the body fit layer, actuators that deliver the therapy, valves that control the inflation level, etc. In such an embodiment the vest is portable. By way of example, this scenario can be applicable to patients who need therapy during the day outside the home. As with certain other embodiments, components or parts of a system may be provided in a modular fashion. For example, a battery may be reversibly attachable to the vest, e.g., via a compartment for housing the battery in the vest with a port or connection to allow the battery to supply power to various components of the vest. In a mobile scenario, a user may add the battery to the vest to power actuator(s) for mobile therapy, whereas in a stationary setting, the battery may or may not be used (and may be removed) in favor of an airflow generator, which may likewise be attached via a suitable port or connection. Such a modular system may therefore offer the benefits of a stationary and mobile vest in a hybrid solution.

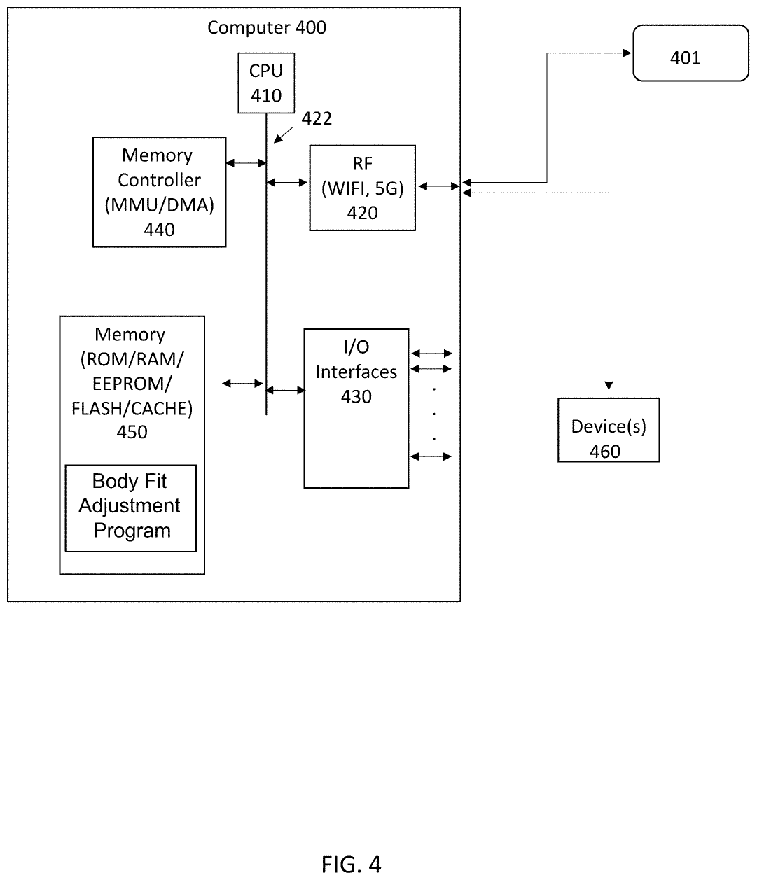

Referring to FIG. 4, it will be readily understood that certain embodiments can be implemented using any of a wide variety of devices or combinations of devices. In FIG. 4 an example system on chip (SoC) included in a computer 400 is illustrated, which may be used in implementing one or more embodiments, e.g., as a controller. The SoC or similar circuitry outlined in FIG. 4 may be implemented in a variety of devices, for example similar circuitry may be included in an air flow generator, such as illustrated in FIG. 1 at 102, in place of CPU 101 in FIG. 1, or another device or platform. In addition, circuitry other than a SoC, an example of which is provided in FIG. 4, may be utilized in one or more embodiments. The SoC of FIG. 4 includes functional blocks, as illustrated, integrated onto a single semiconductor chip to meet specific application requirements.

The central processing unit (CPU) 410, which may include one or more graphics processing units (GPUs) and/or micro-processing units (MPUs), includes an arithmetic logic unit (ALU) that performs arithmetic and logic operations, instruction decoder that decodes instructions and provides information to a timing and control unit, as well as registers for temporary data storage. The CPU 410 may comprise a single integrated circuit comprising several units, the design and arrangement of which vary according to the architecture chosen.

Computer 400 also includes a memory controller 440, e.g., comprising a direct memory access (DMA) controller to transfer data between memory 450 and hardware peripherals. Memory controller 440 includes a memory management unit (MMU) that functions to handle cache control, memory protection, and virtual memory. Computer 400 may include controllers for communication using various communication protocols (e.g., I²C, USB, etc.).

Memory 450 may include a variety of memory types, volatile and nonvolatile, e.g., read only memory (ROM), random access memory (RAM), electrically erasable programmable read only memory (EEPROM), Flash memory, and cache memory. Memory 450 may include embedded programs and downloaded software, e.g., control software such as a body fit adjustment program for operating a body fit layer, a therapy layer, etc. By way of example, and not limitation, memory 450 may also include an operating system, application programs, other program modules, and program data, which may be downloaded, updated, or modified via remote devices.

A system bus 422 permits communication between various components of the computer 400. I/O interfaces 430 and radio frequency (RF) devices 420, e.g., WIFI and telecommunication radios, may be included to permit computer 400 to send and receive data to and from remote devices using wired or wireless mechanisms. The computer 400 may operate in a networked or distributed environment using logical connections to one or more other remote computers or databases. The logical connections may include a network, such local area network (LAN) or a wide area network (WAN), but may also include other networks/buses. For example, computer 400 may communicate data with and between a remote device 460, such as a body scanning device or an electronic medical records system, and another device 401, e.g., a vest or system component including CPU 101 as shown in FIG. 1, etc.

The computer 400 may therefore execute program instructions configured to store and control a body fit layer or components, a therapy layer or components, a combination thereof, and perform other functionality of the embodiments, as described herein. A user can interface with (for example, enter commands and information) the computer 400 through input devices, which may be connected to I/O interfaces 430. A display or other type of device may be connected to the computer 400 via an interface selected from I/O interfaces 430.

It should be noted that the various functions described herein may be implemented using instructions stored on a memory, e.g., memory 450, that are transmitted to and executed by a processor, e.g., CPU 410. Computer 400 includes one or more storage devices that persistently store programs and other data. A storage device, as used herein, is a non-transitory computer readable storage medium. Some examples of a non-transitory storage device or computer readable storage medium include, but are not limited to, storage integral to computer 400, such as memory 450, a hard disk or a solid-state drive, and removable storage, such as an optical disc or a memory stick.

Program code stored in a memory or storage device may be transmitted using any appropriate transmission medium, including but not limited to wireless, wireline, optical fiber cable, RF, or any suitable combination of the foregoing.

Program code for carrying out operations may be written in any combination of one or more programming languages. The program code may execute entirely on a single device, partly on a single device, as a stand-alone software package, partly on single device and partly on another device, or entirely on the other device. In an embodiment, program code may be stored in a non-transitory medium and executed by a processor to implement functions or acts specified herein. In some cases, the devices referenced herein may be connected through any type of connection or network, including a local area network (LAN) or a wide area network (WAN), or the connection may be made through other devices (for example, through the Internet using an Internet Service Provider), through wireless connections or through a hard wire connection, such as over a USB connection.

Example embodiments are described herein with reference to the figures, which illustrate example methods, devices and program products according to various example embodiments. It will be understood that the actions and functionality may be implemented at least in part by program instructions. These program instructions (computer code) may be provided to a processor of a device to produce a special purpose machine, e.g., a controller, such that the instructions, which execute via a processor of the device implement the functions/acts specified.

It is worth noting that while specific elements are used in the figures, and a particular ordering of elements has been illustrated, these are non-limiting examples. In certain contexts, two or more elements may be combined, an element may be split into two or more elements, or certain elements may be re-ordered, re-organized, or omitted, as appropriate, as the explicit illustrated examples are used only for descriptive purposes and are not to be construed as limiting.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A therapy vest, comprising:
a vest body that includes:
one or more body fit compartments; and
one or more therapy compartments;
wherein one or more of the one or more body fit compartments and one or more of the one or more therapy compartments are configured to be independently controllable.

2. The therapy vest of claim 1, comprising a sensor associated with the one or more body fit compartments;
wherein the sensor is configured to provide sensor data to a controller.

3. The therapy vest of claim 1, wherein one or more of the one or more body fit compartments are disposed on top of one or more of the one or more therapy compartments in a state where a patient is wearing the therapy vest.

4. The therapy vest of claim 1, wherein one or more of the one or more body fit compartments are disposed under one or more of the one or more therapy compartments in a state where a patient is wearing the therapy vest.

5. The therapy vest of claim 1, wherein at least one of the one or more body fit compartments is inflatable.

6. The therapy vest of claim 1, wherein the one or more body fit compartments comprise two or more body fit compartments.

7. The therapy vest of claim 6, wherein the two or more body fit compartments are configured for independent control.

8. The therapy vest of claim 6, comprising a separator disposed between the two or more body fit compartments.

9. The therapy vest of claim 8, wherein the separator comprises a valve configured to control fluid communication between the two or more body fit compartments.

10. The therapy vest of claim 1, wherein each of the one or more body fit compartments and the one or more therapy compartments is inflatable.

11. The therapy vest of claim 1, comprising a separator;
wherein the one or more therapy compartments comprise two or more therapy compartments separated by the separator.

12. The therapy vest of claim 1, comprising one or more actuators disposed within the one or more therapy compartments.

13. The therapy vest of claim 1, comprising:
a power source;
a processor; and
one or more sensors;
wherein the processor is configured to control, using sensor data obtained from the one or more sensors, the one or more body fit compartments independent from the one or more therapy compartments.

14. A system, comprising:
an airflow generator; and
a therapy vest, comprising:
a vest body that includes:
one or more body fit compartments; and
one or more therapy compartments;
wherein one or more of the one or more body fit compartments and one or more of the one or more therapy compartments are configured to be independently controllable using airflow from the airflow generator.

15. A system, comprising:
a controller configured to obtain one or more body fit parameters; and
a therapy vest, comprising:
a vest body that includes:
one or more body fit compartments;
one or more sensors; and
one or more therapy compartments;
wherein the one or more body fit compartments are inflated by the controller based on one or more of the one or more body fit parameters and data obtained from the one or more sensors.
